(19) United States
(12) Patent Application Publication
(10) Pub. No.: US 2014/0273062 A1
(43) Pub. Date: Sep. 18, 2014

(54) Cell Pack for the Growth and Manipulation of Three Dimensional Cell Cultures

(71) Applicants: Thomas O'Shaughnessy, Arlington, VA (US); Paul T. Charles, Bowie, MD (US); Kirth Simmonds, Clinton, MD (US); Amit Bagchi, Rockville, MD (US); Ryan Mcculloch, Alexandria, VA (US)

(72) Inventors: Thomas O'Shaughnessy, Arlington, VA (US); Paul T. Charles, Bowie, MD (US); Kirth Simmonds, Clinton, MD (US); Amit Bagchi, Rockville, MD (US); Ryan Mcculloch, Alexandria, VA (US)

(73) Assignee: The Government of the United States of America, as represented by the Secretary of the Navy, Arlington, VA (US)

(21) Appl. No.: 14/197,724

(22) Filed: Mar. 5, 2014

Related U.S. Application Data
(60) Provisional application No. 61/777,899, filed on Mar. 12, 2013.

(51) Int. Cl. C12M 1/12

(52) U.S. Cl. CPC C12M 37/02
USPC 435/29; 435/297.1; 435/395

(57) ABSTRACT

Described herein is a sealed cell pack with a permeable membrane for growth and manipulation of three-dimensional cell cultures. This allows a cell culture to be removed from the laboratory and subjected to real world insults before being returned to culture conditions for continued growth and study. One application is for use in the study of the direct effects of blast waves on neuronal cells and methods for mitigating this response.

### CROSS-REFERENCE TO RELATED APPLICATIONS

This application claims the benefit of U.S. Provisional Application 61/777,899 filed on Mar. 12, 2013, the entirety of which is incorporated herein by reference.

### BACKGROUND

Traditionally, cell culture has been confined to the laboratory with cells cultured, grown, and tested under sterile conditions. Typical culture containers are small plastic dishes (most commonly 35-100 mm in diameter), flat walled flasks (most commonly with surface areas of 25 or 75 cm²), or 6-96 well multi-well plates. In all cases, adherent cells are grown on the bottom surfaces of the culture plastic covered with growth media. Cells that form tight mono-layers, such as endothelial or epithelial cells, are often grown on cell culture inserts; a plastic well with a bottom formed from a membrane permeable to the culture media such as cellulose acetate or track etch polycarbonate. These inserts sit in the well of a multi well plate and provide two distinct sides for the mono-layer of cells. Different media can be placed above and below the cells or the top of the cells can be exposed to atmosphere (again the atmosphere must be sterile; mimics the air-cell interface of the lung epithelium for example).

Researchers have previously used pouches made from track-etched polycarbonate with 0.4 μm pores to implant cells in an animal, while keeping them separate and contained (Gates and Lazarus, 1977). The cells were grown in suspension within the pouch and the porous membrane allowed the secretions of the cells to enter the animals system. This eliminated the issues with the isolated cells being destroyed by the animal's immune system.

Traditional cell culture is two-dimensional with cells growing on a flat surface. This is not physiological, and more recently researchers have been mimicking the three-dimensional matrix that cells grow in to better reproduce the in vivo cellular environment under in vitro conditions. For example, it has been shown that primary murine neurons can be grown in a three-dimensional collagen matrix in a 24 well plate (O'Conner et al., 2001) and that these neurons form functional synapses with each other (O'Shaughnessy et al., 2003). By combining three-dimensional culture techniques with a porous membrane having pores small enough to maintain sterility, a cell culture system can be developed that can be removed from the laboratory and subjected to real world insults before being returned to culture conditions for continued growth and study.

A need exists for technology enabling three-dimensional cell cultures to be removed from the standard culture environment for short periods of time while maintaining sterility of the cells.

### BRIEF SUMMARY

In a first embodiment, a sealed cell pack includes a ring having an inner diameter, a thickness defined by distance between first and second opposing faces of the ring, and an interior volume defined by the thickness and the inner diameter of the ring; a porous membrane bonded to the first face of the ring, the porous membrane comprising pores sufficiently small to exclude passage of organisms and sufficiently large to permit passage of nutrients, waste, and dyes/stains; and a transparent film bonded to the second face of the ring, wherein the ring either (1) is comprised of elastomer or (2) includes a sealable orifice suitable for introduction of cells and growth media, wherein said interior volume is in a sterilized condition, and wherein the thickness of the ring is sufficiently small to allow diffusion of nutrients through the porous membrane to permit cell growth throughout said interior volume.

In a second embodiment, a sealed cell pack includes a ring having an inner diameter, a thickness defined by distance between first and second opposing faces of the ring, and an interior volume defined by the thickness and the inner diameter of the ring; a first porous membrane bonded to the first face of the ring; and a second porous membrane bonded to the second face of the ring; wherein the first and second porous membranes comprise pores are sufficiently small to exclude passage of organisms and sufficiently large to permit passage of nutrients and waste, wherein the ring either (1) is comprised of elastomer or (2) includes a sealable orifice suitable for introduction of cells and growth media, wherein said interior volume is in a sterilized condition, and wherein the thickness of the ring is sufficiently small to allow diffusion of nutrients through the first and second porous membranes to permit cell growth throughout said interior volume.

Further embodiments include using the cells packs of either the first or second embodiments by introducing living cells and a three-dimension matrix suitable for cellular growth into the interior volume.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows cell packs in various states of assembly according to exemplary embodiments.

FIG. 2 shows confocal images of the interior of a cell pack containing fluorescent beads imaged from both sides.

FIG. 3 illustrates a cell pack containing a 3D neuronal culture sealed with sterile vacuum grease.

FIG. 4 shows confocal imaging results of live/dead staining of cells in a cell pack after 25 days in culture.

FIG. 5 illustrates alternative embodiments having rings made of elastomer.

### DETAILED DESCRIPTION

### Definitions

Before describing the present invention in detail, it is to be understood that the terminology used in the specification is for the purpose of describing particular embodiments, and is not necessarily intended to be limiting. Although many methods, structures and materials similar, modified, or equivalent to those described herein can be used in the practice of the present invention without undue experimentation, the preferred methods, structures and materials are described herein. In describing and claiming the present invention, the following terminology will be used in accordance with the definitions set out below.

As used in this specification and the appended claims, the singular forms “a”, “an,” and “the” do not preclude plural referents, unless the content clearly dictates otherwise.

As used herein, the term “and/or” includes any and all combinations of one or more of the associated listed items.

As used herein, the term “about” when used in conjunction with a stated numerical value or range denotes somewhat more or somewhat less than the stated value or range, to within a range of ±10% of that stated.

### Description

Described herein is a sealed cell pack with a nutrient/dye permeable membrane for growth and manipulation of three-dimensional cell cultures. One application is for use in the study of the direct effects of blast waves on neuronal cells via shock tubes.

The cell pack includes a porous membrane that allows for nutrients to diffuse into the chamber allowing for maintenance of the cells by standard culture methods. Such pores would also permit the passage of most dyes and stains. The pores of the membrane are small enough that sterility is maintained within the chamber allowing for the cell chamber to be placed into environments for testing that would not normally be possible with standard cell culture. For example, the cell pack can be removed from the incubator/culture media for a short time to be exposed to simulated blast waves generated by a shock tube. It is also possible to stain and image the cell culture while within the cell pack, thus maintaining the original dimensions and orientation of the cell culture within the cell pack.

In an exemplary reduction to practice, murine cortex cells were seeded into collagen and loaded into several sealed cell packs with a clear polystyrene membrane on one side and a track etch 0.22 micron pore polycarbonate membrane on the other. After 25 days, a standard live/dead stain was performed on one of the cell packs while it was still sealed. Live cells were clearly visible in the sealed pack as observed by fluorescent microscopy.

Exemplary cell packs are described: they comprise a plastic ring with an inner diameter of 0.45 inch and an outer diameter of 0.65 inch. The ring is 0.15 inch thick and milled from either food grade polystyrene or acrylic. A 0.04 inch hole was drilled in the side of the ring to allow filling once the cell pack was assembled. A 50 μm thick film of polystyrene was glued onto one side of the ring using Kwik-Cast glue (World Precision Instruments, Sarasota, Fla.) which was tested and found to be non-toxic to the primary neuronal cultures used to test the cell packs. A 10 micron thick track etch polycarbonate (TEPC) membrane with 0.22 μm pores (pore density 3×10⁸ pores/cm²; Structure Pore, Inc., West Chester, Pa.) was glued to the other side of the ring with the Kwik-Cast glue. Once assembled the glue was allowed to dry and the cell packs were soaked in water for 12-24 hours to remove any soluble agents from the plastics and glue, dried, and stored for use.

FIG. 1 shows cell packs in various states of assembly according to exemplary embodiments. The two left most rings are “open” cell packs which lacked the TEPC membrane and were used for early testing. The right most ring is an early “closed” cell pack. The TEPC is on the bottom in the case and can be seen as a white layer through the upper transparent polystyrene film. At the roughly one o'clock position the fill hole can be seen through the translucent ring of the cell pack. The tinting may be seen from the Kwik-Cast glue. Total volume of the cell pack is 0.4 mL.

Before placing cells in the cell pack, the pack should be sterilized. This may accomplished by filling the cell pack with 70% ethanol and then submersing it in the same for 15 minutes. Other forms of sterilization, for example irradiation, are also possible. After 15 minutes, the pack is removed from the ethanol, emptied, and allowed to dry. The cell packs may be filled by making a collagen gel mix and combining it with the cells to be cultured, then injecting the mixture into the cell pack with a sterile syringe and needle. Once the pack is filled, the opening is preferably sealed with a small amount of sterile silicon vacuum grease and the cell pack is placed into culture medium (in a dish or flask) and placed in a standard CO₂ incubator where the collagen gel forms around the cells. The cell packs can be placed in separate dishes or multiple cell packs can be placed in a single dish or flask. Media can be changed at regular intervals as with standard culture as the media is free to diffuse into the cell packs through the TEPC membrane and waste products from the cells diffuse out of the cell pack. The size of the cell pack was optimized to ensure that the cells furthest away from the TEPC membrane receive sufficient nutrients to survive. This consideration limits the maximum thickness of the cell packs. When the cell packs are sealed after being loaded with cells, they can be removed from the culture media for a short time without compromising the cell culture. The pores in the TEPC membrane are too small to allow organisms to enter the cell pack, thus keeping them sterile. Prior to return to sterile culture, the exterior of the cell pack should be washed sufficiently with sterile media or phosphate buffered saline to remove possible contamination from the exterior, otherwise there is a risk of contaminating growth media outside the cell pack.

### Cell Pack Testing

A cell pack was tested to determine if a collagen hydrogel could be formed inside the cell pack and if the hydrogel would adhere to the walls and remain immobilized, similar to what is seen in a 24 well culture plate. For these tests, cell packs with two polystyrene membranes (no TEPC) were used to allow imaging of the internal collagen gel from both sides of the cell pack using a confocal microscope. The cell packs were filled with a collagen gel containing a small number of 10-14 μm fluorescent yellow polystyrene beads (Spherotech, Lake Forest, Ill.). Three-dimensional images of the cell pack were then taken from both sides using a confocal microscope and the images compared to determine if the beads were immobilized. FIG. 2 shows confocal images of a cell pack containing fluorescent beads imaged from both sides. Between imaging the two sides the cell pack was manipulated in number of ways (inverted, rotated, etc.). Each set of images shows the XY plane in the top left, the YZ plane in the top right, and the XZ plane in the lower panel. Note that the Z and X axes are flipped between the image sets (axis arrows always point in positive direction), but that the identified particles maintain the same relative positions. The results demonstrated that the collagen gel was indeed immobilized within the cell pack.

Testing then moved on to living cell cultures. The first tests of a cell pack for a cell culture utilized a cellulose acetate membrane instead of TEPC. The cell packs were loaded with neurons derived from a combined murine cortex/hippocampus/subventricular zone brain section. The cells were loaded at a density of 1.1×10⁶ cells/mL in a collagen mix using NB/B27 media to suppress glial cell growth. For these initial viability tests the fill holes of the cell packs were not sealed. The cell packs were dissembled at several time points and standard live/dead stains (calcein-AM/ethidium homodimer-1) performed on the recovered collagen gels to look for live neurons. Substantial numbers of live neurons were found in all cases up to the longest time point tested, 67 days in a cell pack.

The next set of tests was with a working cell pack design as described herein. This includes the TEPC membrane and sealing of the cell packs with vacuum grease. For these experiments, neuronal cells were isolated from murine brain cortex. Type IV rat tail collagen (100 mg; Roche, Basel, Switzerland) was dissolved in 5 mL of 0.2% sterile acetic acid and placed on ice. Cells were isolated by a standard mechanical dissociation of the cortex tissue, suspended in DMEM culture media supplemented with B27 (Invitrogen, Carlsbad, Calif.), 5% horse serum and 5% fetal bovine serum at a density of 2.4-2.8×10⁶ cells/mL. One milliliter of the collagen solution was then mixed with 1 mL of 2× phosphate-buffered saline (2×PBS) and 10 μL of 1.3 M NaOH to neutralize the acetic acid and allow gelling of the collagen. Finally, 2 mL of the cell suspension was mixed with the above and immediately injected into the cell packs.

The cell packs were sealed with sterile vacuum grease and then placed into sterile dishes that were specially made to fit the cell packs and support culture with only 2 mL of additional media. FIG. 3 shows a sealed cell pack containing murine cortex neurons in a collagen gel. This cell pack was assembled with a variation of the Kwik-Cast glue that is clear rather than green. The dishes with the cell packs were placed into the incubator for one hour to allow the gels to set, and then 2 mL of additional media was added to each cell pack/dish and the dishes returned to the incubator. The cell cultures were ‘fed’ Monday, Wednesday, and Friday by a 50% change of bathing media with DMEM supplemented only with B27 until testing. Testing involved performing a live/dead stain on the cells while they remained in the cell packs and then imaging with a confocal microscope (the dyes diffused through the TEPC membrane, staining and imaging was all done on the sealed cell pack). Viable neural cells were seen in all cell packs tested out to the longest time tested, 30 days in culture. The cultures were comparable to what we have previously seen doing standard 3D cultures in a 24 well plate. Immobilization of the cells and gels within the cell pack was also noted. FIG. 4 shows confocal images of neurons and glia in a cell pack after 25 days in culture. The axes/scale bars indicate the plane of each image. The quantity of dead cells (green=live; red=dead) is typical for this type of culture as not all cells survive the disassociation process. Note that axons and dendrites from the neurons are clearly visible in the XY plane (top down view).

Additional testing involved taking one cell pack out of the sterile culture environment, subjecting it to a mechanical challenge, returning it to culture, and finally demonstrating survival of the cells. For this test the cell pack was removed from the culture dish and placed in a small Ziploc bag for transport between buildings housing required equipment. The cell pack was placed between layers of gel-based brain simulant material and then positioned in front of a shock tube (PCB Piezotronics, Depew, N.Y.). Shock tubes use compressed gas to create pressure waves that simulate a blast wave front. For this test the cell pack was exposed to a pressure wave front that was approximately 1 ms in duration and peaked at 40 psi in the air just prior to the front brain simulant layer. The cell pack was removed from the rig and examined for damage. No damage was noted. The cell pack was then returned to the culture lab in another building, rinsed thoroughly in sterile Dulbecco's phosphate-buffered saline (DPBS), and returned to its culture dish and the incubator. The following day no evidence of contamination was noted in the cell pack. The cell pack was live/dead stained as before and imaged on the confocal microscope. As before, an immobilized collagen gel was found containing a live culture of neurons and glia.

There are a number of modifications or additions that could be implemented to improve or augment the function of the cell packs. For example if a larger thickness is desired and imaging within the cell pack is not required the transparent polystyrene film could be replaced with a second porous membrane which would allow nutrients to enter the cell pack from both sides and effectively double the thickness that could be supported. Moreover, by constructing the ring from rubber or another suitable elastomer instead of plastic (FIG. 5), no fill hole would be required as the rubber-based cell pack could be filled by penetrating the rubber with a hypodermic needle; the rubber would seal back up with the removal of the needle. FIG. 5 shows examples of rubber-based cell packs next to a standard cell pack.

There are also a number of ways to instrument the cell pack. For example, an electrode array could be constructed on the polystyrene membrane for recording the electrical signals of the neurons (Gross et al., 1985). Alternatively a layer of PVDF could be added to the polystyrene film to allow for measurement of pressure waveforms passing through the cell pack (Shirinov and Schomburg, 2008).

### Advantages

Described herein is a technique for packaging three-dimensional cell cultures for use outside the laboratory by placing them inside a sealed, sterile chamber that allows for diffusion of gases, nutrients, drugs, and dyes/stains into and out of the chamber. Cells can not only be exposed to environmental conditions while in the protective chamber, but many traditional tests and stains can be performed on the cells while still within the sealed cell pack. Finally, use of a transparent polystyrene film allows imaging of the cell culture while still within the cell pack.

Existing commercial systems available do not appear to provide a similar functionality to what is described in this document. While it is possible to seal some culture dishes/flasks for removal from the culture environment, the volumes are typically too large for experimental purposes, and doing this multiple times while maintaining sterility can be challenging. Finally, the cells are not able to be exposed to environmental molecules as the culture system would be sealed and lack a permeable membrane.

### Concluding Remarks

All documents mentioned herein are hereby incorporated by reference for the purpose of disclosing and describing the particular materials and methodologies for which the document was cited.

Although the present invention has been described in connection with preferred embodiments thereof, it will be appreciated by those skilled in the art that additions, deletions, modifications, and substitutions not specifically described may be made without departing from the spirit and scope of the invention. Terminology used herein should not be construed as being “means-plus-function” language unless the term “means” is expressly used in association therewith.

### REFERENCES

1. Bader A. Device for the treatment of cell cultures. U.S. Pat. No. 5,658,797.
2. Brauker et al. Implanted tumor cells for the prevention and treatment of cancer. U.S. Pat. No. 6,156,305.
3. Pourahmadi et al. Integrated fluid manipulation cartridge. U.S. Pat. No. 6,440,725.
4. Toner et al. Cell culture systems and methods for organ assist devices. U.S. Pat. No. 6,759,245
5. Martin G R and Tanner A J. Cell culture apparatus for co-culture of cells. United States Patent Application Publication 2008/0299649
6. Gates R J and Lazarus N R. (1977) “Reversal of streptozotocin-induced diabetes in rats by intraperitoneal implantation of encapsulated neonatal rabbit pancreatic tissue.” The Lancet 310: 1257-1259.
7. Gross G W, Wen W, Lin J, Journal of Neuroscience Methods 15, 243 (1985).
8. O'Conner S M, Stenger D A, Shaffer K M, Ma W, Neuroscience Letters 304, 189 (2001).
9. O'Shaughnessy T J, Lin H J, Ma W, Neuroscience Letters 340, 169 (2003).
10. Shiribov A V, Schomburg W K, Sensors and Actuators A 142, 48 (2008).
11. Vaage J and Harlos J P. (1991) “Collagen production by macrophages in tumour encapsulation and dormancy.” British Journal of Cancer 63: 758-762

What is claimed is:

1. A sealed cell pack comprising:
a ring having an inner diameter, a thickness defined by distance between first and second opposing faces of the ring, and an interior volume defined by the thickness and the inner diameter of the ring;
a porous membrane bonded to the first face of the ring, the porous membrane comprising pores sufficiently small to exclude passage of organisms and sufficiently large to permit passage of nutrients and waste; and
a transparent film bonded to the second face of the ring,
wherein the ring either (1) is comprised of elastomer or (2) includes a sealable orifice suitable for introduction of cells and growth media,
wherein said interior volume is in a sterilized condition, and
wherein the thickness of the ring is sufficiently small to allow diffusion of nutrients through the porous membrane to permit cell growth throughout said interior volume.

2. The sealed cell pack of claim 1, further comprising a three-dimensional matrix suitable for cellular growth disposed within the interior volume, and living cells growing among the three-dimensional matrix.

3. The sealed cell pack of claim 2, wherein said three-dimensional matrix is collagen.

4. The sealed cell pack of claim 2, wherein said living cells comprise neuronal cells.

5. The sealed cell pack of claim 1, wherein said interior volume is about 0.4 mL.

6. The sealed cell pack of claim 1, further comprising an instrument attached thereto.

7. The sealed cell pack of claim 5, wherein said instrument comprises an electrode array or a pressure-sensitive layer.

8. The sealed cell pack of claim 1, wherein said porous membrane comprises polycarbonate and said pores are 0.22 microns.

9. A sealed cell pack comprising:
a ring having an inner diameter, a thickness defined by distance between first and second opposing faces of the ring, and an interior volume defined by the thickness and the inner diameter of the ring;
a first porous membrane bonded to the first face of the ring; and
a second porous membrane bonded to the second face of the ring;
wherein the first and second porous membranes comprise pores sufficiently small to exclude passage of organisms and sufficiently large to permit passage of nutrients and waste,
wherein the ring either (1) is comprised of elastomer or (2) includes a sealable orifice suitable for introduction is cells and growth media,
wherein said interior volume is in a sterilized condition, and
wherein the thickness of the ring is sufficiently small to allow diffusion of nutrients through the first and second porous membranes to permit cell growth throughout said interior volume.

10. The sealed cell pack of claim 9, further comprising a three-dimension matrix suitable for cellular growth disposed within the interior volume, and living cells growing among the three-dimensional matrix.

11. The sealed cell pack of claim 10, wherein said living cells comprise neuronal cells.

12. The sealed cell pack of claim 10, wherein said three-dimensional matrix is collagen.

13. The sealed cell pack of claim 9, wherein said interior volume is about 0.4 mL.

14. The sealed cell pack of claim 9, further comprising an instrument attached thereto.

15. The sealed cell pack of claim 14, wherein said instrument comprises an electrode array or a pressure-sensitive layer.

16. The sealed cell pack of claim 9, wherein both said porous membranes comprise polycarbonate and said pores are—0.22 microns.

17. A method of using a sealed cell pack, the method comprising:
providing a sealed cell pack comprising
a ring having an inner diameter, a thickness defined by distance between first and second opposing faces of the ring, and an interior volume defined by the thickness and the inner diameter of the ring;
a porous membrane bonded to the first face of the ring, the porous membrane comprising pores sufficiently small to exclude passage of organisms and sufficiently large to permit passage of nutrients and waste; and
a transparent film bonded to the second face of the ring,
wherein the ring either (1) is comprised of elastomer or (2) includes a sealable orifice suitable for introduction of cells and growth media,
wherein said interior volume is in a sterilized condition, and
wherein the thickness of the ring is sufficiently small to allow diffusion of nutrients through the porous membrane to permit cell growth throughout said interior volume; and
introducing living cells and a three-dimension matrix suitable for cellular growth into the interior volume.

18. The method of claim 18, further comprising visualizing said living cells through said transparent film while maintaining sterile culture conditions.

19. The method of claim 18, further comprising applying a stain or dye to said living cells through said porous membrane.
